(19) United States
(12) Patent Application Publication
(10) Pub. No.: US 2022/0125916 A1
(43) Pub. Date: Apr. 28, 2022

(54) PEANUT HYPOALLERGENIC FORMULATIONS FOR DETERMINING THE RISK OF ANAPHYLAXIS

(71) Applicant: THE ROYAL INSTITUTION FOR THE ADVANCEMENT OF LEARNING/MCGILL UNIVERSITY, Montréal (CA)

(72) Inventors: Casey COHEN, Montréal (CA); Bertrand J. JEAN-CLAUDE, Montréal (CA); Bruce MAZER, Cote Saint-Luc (CA)

(21) Appl. No.: 17/510,772

(22) Filed: Oct. 26, 2021

(30) Foreign Application Priority Data
Oct. 26, 2020 (AU) 2020260379
Oct. 26, 2020 (CA) 3097204

(51) Int. Cl. A61K 39/35; A61K 9/00; A61P 37/08

(52) U.S. Cl. CPC A61K 39/35; A61P 37/08; A61K 9/0021

(57) ABSTRACT

The present disclosure provides a method for stratifying subjects with respect to their relative risk of developing anaphylaxis upon the ingestion of a peanut allergen. The method is based on the differential allergic response of the subject in a skin prick test using an Ara h 2 hypoallergenic formulation (which substantially lacks or is devoid of Ara h 8). The present disclosure also provides a peanut hypoallergenic formulation, which can be used as a reagent during the skin prick test, as an oral immunotherapy reagent or as a food product. The present disclosure further provides a process for making the peanut hypoallergenic formulation.

### TECHNOLOGICAL FIELD

The present disclosure concerns allergenic formulations that can be used to determine the risk of developing anaphylaxis in a subject after the consumption of a peanut or a peanut allergen.

### BACKGROUND

Peanut allergy is extremely common, affecting approximately 1.5% of children in North America, Australia and the UK. It is an extremely important cause of anaphylaxis and utilization of hospital emergency room resources. Most individuals with peanut allergy are not treated; rather they strictly avoid peanut-containing foods and carry precautionary injected epinephrine in case of accidental ingestion. More recently, clinical trials in oral immunotherapy (OIT), exposing peanut-allergic individuals to small incremental doses of peanut, have shown promising results, leading to the first ever US FDA-approved peanut OIT treatment, Palforzia™. However, this therapy does not lead to complete tolerance, specifically the ability to eat all forms and amounts of peanuts securely. At best, peanut OIT has improved safety in individuals, but they must ingest consistent small doses of peanut to maintain a level of protection. In addition, there is a significant risk of allergic reactions during therapy, including anaphylaxis, and the debate is ongoing over whether it is appropriate to provide this therapy outside the context of well-controlled trials.

In food allergies such as egg and milk, the rate of spontaneous resolution is considerably higher than for peanut. Indeed, children with egg or milk allergy can frequently introduce small amounts of well-cooked egg or milk into their diets safely as they grow. Natural history studies of this practice have indicated that the patients are able to increase the cooked form of the allergen into their diets and ultimately a significant number evolve to complete tolerance. Normal cooking processes denature or linearize egg or milk proteins, which may explain their decreased allergenicity, and prolonged frequent exposure may act as a form of OIT, albeit with more safety than conventional OIT.

Peanut does not appear to denature under normal cooking conditions. Structural biology analyses have focused on the three-dimensional structure of the major peanut protein allergens and recent reports have thoroughly described their X-ray structures. This class of proteins is rich in disulfide bridges, which explains their resistance to denaturation at high temperature. In fact, glycation at high temperature is proposed to be a primary mechanism of enhancement of allergenic responses to peanut, as shown by quantification of IgE-binding. Glycation primarily results from the Maillard reaction, an addition of amines on reducing sugars to provide Schiff bases that rearrange to form a wide range of products, of which the Advanced Glycation End-products (AGE) are believed to be of relevance to allergenicity. Importantly, although the molecular composition of the peanut (i.e. proteins, amino acids, metal ion, sugar content) is now well known, the specific contribution of free sugars and amino acids to the enhancement of allergenicity of peanuts at high temperatures has yet to be defined.

Previous studies suggest a decrease in IgE-binding in boiled and fried peanuts when compared with raw. It has been reported that low-molecular-weight proteins are transferred from the peanuts into the cooking water throughout boiling, particularly the 2S albumins Ara h 2, Ara h 6 and Ara h 7, potentially explaining a decrease in IgE-binding. Moreover, it has also been found that autoclaving roasted peanuts produces a decrease of IgE-binding capacity of peanut allergens and in wheal size by skin prick test, as well as the unfolding of proteins and reduction in overall secondary structure as shown by circular dichroism (CD) experiments (Cabanillas et al., 2012).

There is a need in the art to be able to stratify subjects allergic to peanuts with respect to their relative risk of developing anaphylaxis. There is also a need in the art to be provided with a hypoallergenic peanut composition for providing a safer oral immunotherapy.

### BRIEF SUMMARY

The present disclosure provides a method for stratifying subjects with respect to their relative risk of developing anaphylaxis upon the ingestion of a peanut allergen. The method is based on the differential allergic response of the subject in a skin prick test using a Ara h 2 allergen (which substantially lacks or is devoid of Ara h 8).

According to a first aspect, the present disclosure provides a process for making a peanut hypoallergenic formulation. The process comprises (a) providing an initial composition comprising a Ara h 2 allergen and a Ara h 8 allergen; and (b) submitting the initial composition to a simultaneous heat treatment and pressure treatment to obtain the peanut hypoallergenic formulation, wherein the peanut hypoallergenic formulation substantially lacks the Ara h 8 allergen. In an embodiment, the initial composition comprises or is derived from a whole peanut. In another embodiment, the heat treatment (of the simultaneous heat treatment and pressure treatment) comprises heating the initial composition to a temperature between 120° C. and 140° C. In another embodiment, the pressure treatment (of the simultaneous heat treatment and pressure treatment) comprises applying a pressure between 1.15 and 2.60 atm to the initial composition. In still another embodiment, the process comprises submitting the initial composition to the simultaneous heat and pressure treatment for a period between about 2 to about 30 minutes. In still another embodiment, the pressure treatment (of the simultaneous heat treatment and pressure treatment) comprises applying a vapor pressure to the initial composition. In some embodiments, step (b) comprises autoclaving the initial composition. In additional embodiments, the process further comprises cooking, pureeing, extruding and/or making a flour of the initial composition, the peanut hypoallergenic formulation, or both. In still further embodiments, the process comprises formulating the peanut hypoallergenic formulation in a skin prick test reagent, an oral immunotherapy reagent or a food product. In specific embodiments, the peanut hypoallergenic formulation has a ¹H nuclear magnetic resonance CH NMR) spectrum profile at least substantially similar or identical to the ¹H NMR spectrum profile shown in FIG. 10C in the range between 0.5 and 2 ppm and between 6.5 and 8.5 ppm. In some embodiments, the process further comprises performing a ¹H nuclear magnetic resonance CH NMR) analysis of the initial composition, the peanut hypoallergenic formulation or both. In some embodiments, the ¹H nuclear magnetic resonance CH NMR) analysis is a solution ¹H NMR or a high-resolution magic angle spinning (HR-MAS)¹H NMR analysis.

In a second aspect, the present disclosure provides a peanut hypoallergenic formulation comprising a Ara h 2 allergen and substantially lacking a Ara h 8 allergen. In some embodiments, the peanut hypoallergenic formulation has a ¹H nuclear magnetic resonance CH NMR) spectrum profile at least substantially similar or identical to the ¹H NMR spectrum profile shown in FIG. 10C in the range between 0.5 and 2 ppm and between 6.5 and 8.5 ppm. In yet additional embodiments, the peanut hypoallergenic formulation is obtainable or obtained by the process described herein.

In a third aspect, the present disclosure provides a skin prick test reagent comprising the peanut hypoallergenic formulation described herein.

In a fourth aspect, the present disclosure provides an immunotherapy reagent comprising the peanut hypoallergenic composition described herein.

In a fifth aspect, the present disclosure provides a food product comprising the peanut hypoallergenic formulation described herein. In some embodiment, the food product is a hypoallergenic peanut butter.

In a sixth aspect, the present disclosure provides kit for performing a skin prick test. The kit comprises a first reagent comprising a Ara h 2 allergen and substantially lacking or devoid of a Ara h 8 allergen; and instructions to use the reagent in the skin prick test. In an embodiment, the first reagent is a substantially purified Ara h 2 allergen, the peanut hypoallergenic formulation described herein or the skin prick reagent described herein. In some embodiments, the kit further comprises a second reagent comprising a Ara h 8 allergen. In some embodiments, the second reagent is a substantially purified Ara h 8 allergen or a product derived from a peanut. In some specific embodiments, the second reagent is a whole peanut protein extract. In yet a further embodiment, the kit further comprises the oral immunotherapy reagent described herein.

In a seventh aspect, the present disclosure provides a method for stratifying the risk of a subject to develop anaphylaxis upon the ingestion of a peanut allergen. The method comprises pricking the skin of the subject at a first location to make a first skin abrasion; contacting the first reagent described herein with the first skin abrasion; and determining if the first reagent caused a wheal and flare response towards the first reagent (at the first location). The presence of the wheal and flare response towards the first reagent is indicative that the subject has an increased risk of developing anaphylaxis upon the ingestion of the peanut allergen when compared to a first control subject that did not exhibit a wheal and flare in response to the first reagent. In some embodiments, the method further comprises pricking the skin of the subject at a second location to make a second skin abrasion; contacting the second reagent described herein with the second skin abrasion; and determining if the second reagent caused a wheal and flare response towards the second reagent (at the second location). The presence of the wheal and flare response towards the second reagent, but not towards the first reagent, is indicative that the subject has a decreased risk of developing anaphylaxis upon the ingestion of the peanut allergen when compared to a second control subject that exhibited a wheal and flare towards the first reagent. Alternatively or in combination, the subject was previously determined to exhibit a wheal and flare response towards the second reagent. In some embodiments, the method can be used for screening for subjects suitable for an oral immunotherapy. In some embodiments, the method can include administering the oral immunotherapy to the subject whose risk to develop anaphylaxis has been stratified. In such embodiment, the method can comprise orally administering the oral immunotherapy reagent described herein or the food product described herein to the subject.

In an eighth aspect, the present disclosure provides the use of a first reagent described herein, optionally in combination with the second reagent described herein, to stratify the risk of a subject to develop anaphylaxis upon the ingestion of a peanut allergen. The present disclosure provides the use of the kit described herein to stratify the risk of a subject to develop anaphylaxis upon the ingestion of a peanut allergen. The first reagent is suitable or formulated for contact with a first skin abrasion present on the subject. The second reagent is suitable or formulated for contact with a second skin abrasion present on the subject. The presence of the wheal and flare response towards the first reagent is indicative that the subject has an increased risk of developing anaphylaxis upon the ingestion of the peanut allergen when compared to a first control subject that did not exhibit a wheal and flare in response to the first reagent. The presence of the wheal and flare response towards the second reagent, but not towards the first reagent, is indicative that the subject has a decreased risk of developing anaphylaxis upon the ingestion of the peanut allergen when compared to a second control subject that exhibited a wheal and flare towards the first reagent. In some embodiments, the subject was previously determined to exhibit a wheal and flare response towards the second reagent. In some embodiments, the first reagent and optionally the second reagent can be used for screening for subjects suitable to start and/or continue an oral immunotherapy. In some embodiments, the first reagent and optionally the second reagent can be used with the oral immunotherapy reagent described herein or the food product described herein.

### BRIEF DESCRIPTION OF THE DRAWINGS

Having thus generally described the nature of the invention, reference will now be made to the accompanying drawings, showing by way of illustration, a preferred embodiment thereof, and in which:

FIGS. 1A to 1C show a Western Blot following SDS PAGE using various antibodies. Lanes correspond to the processing conditions as follows: M=Molecular weight marker, 1=Raw, 2=Roast, 3=Autoclave, 4=Autoclave then Roasted, 5=Roasted then Autoclaved.

(FIG. 1A) provides the results obtained with antibodies specific for Ara h 1.

(FIG. 1B) provides the results obtained with antibodies specific for Ara h 2.

(FIG. 1C) provides the results obtained with antibodies specific for Ara h 8.

FIGS. 2A and 2B show the relative quantification of Ara h 2 and Ara h 8 by ELISA. Processing conditions as follows: ●=raw, ▪=roasted, and ▴=autoclaved. Optical Density (OD) values were measured at 450 nm and referenced at 570 nm.

(FIG. 2A) provides the results for Ara h 2. Plates were coated with a maximum concentration of 1 μg/mL peanut protein.

(FIG. 2B) provides the results for Ara h8. Plates were coated with a maximum concentration of 1 mg/mL peanut protein.

FIG. 3 shows a peanut-specific IgE ELISA using the serum of 4 highly allergic subjects. Optical Density (OD) values at 450 nm, referenced at 570 nm, were normalized to the corresponding raw values. Auto-Roast=Autoclaved, then roasted. Roast-Auto=Roasted, then autoclaved. n=4 patients. ns=not significant. ****: p<0.0001, one-way ANOVA, Tukey's multiple comparisons test.

FIGS. 4A to 4C show the solution ¹H NMR spectra of various solutions.

(FIG. 4A) provides the results of a roasted peanut solution.

(FIG. 4B) provides the results of an autoclaved peanut solution.

(FIG. 4C) provides the results of a peanut-soaked solution.

FIGS. 5A and 5B show scatterplots displaying total peanut-specific IgE measured by ELISA versus wheal diameter using different extracts as measured by SPT. Wheal diameters under 3 mm (horizontal line) are clinically insignificant.

(FIG. 5A) provides the results obtained using a commercial standard peanut extract.

(FIG. 5B) provides the results obtained using an autoclaved peanut extract.

FIGS. 6A to 6F shows scatterplots displaying different specific IgE measured by ELISA versus wheal diameter using the different peanut extracts as measured by SPT. Wheal diameters under 3 mm (horizontal line) are clinically insignificant.

(FIG. 6A) provides the results for Ara h 1 using the commercial standard peanut extract.

(FIG. 6B) provides the results for Ara h 1 using the autoclaved peanut extract.

(FIG. 6C) provides the results for Ara h 2 using the commercial standard peanut extract.

(FIG. 6D) provides the results for Ara h 2 using the autoclaved peanut extract.

(FIG. 6E) provides the results for Ara h 8 using the commercial standard peanut extract.

(FIG. 6F) provides the results for Ara h 8 using the autoclaved peanut extract.

FIGS. 7A and 7B shows the HR-MAS ¹H NMR spectra of different peanut preparations.

(FIG. 7A) provides the results obtained with raw peanuts.

(FIG. 7B) provides the results obtained with roasted peanuts.

FIGS. 8A and 8B shows the HR-MAS ¹H NMR spectra of different peanut preparations.

(FIG. 8A) provides the results obtained with whole, intact raw peanuts.

(FIG. 8B) provides the results obtained with raw peanut flour defatted with hexane. Inset in shows spectrum of defatted peanut at 10× magnification.

FIG. 9 shows the solution ¹H NMR spectra of 6 whole raw peanuts soaked in distilled water for 48 hours and the resulting solution was analyzed.

FIGS. 10A to 10C show the solution ¹H NMR spectra of various peanut preparations.

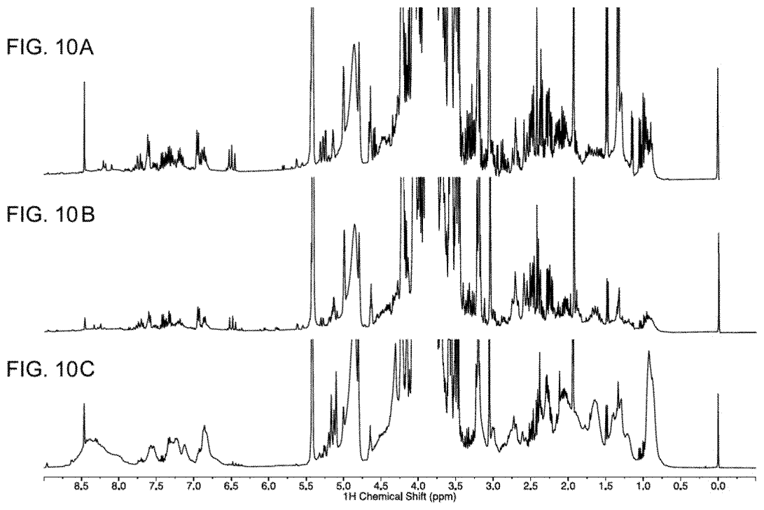

(FIG. 10A) provides the results obtained with 6 whole raw peanuts which have been soaked in distilled water for 48 hours.

(FIG. 10B) provides the results obtained with 6 roasted peanuts which have been soaked in distilled water for 48 hours.

(FIG. 10C) provides the results obtained with 6 autoclaved peanuts which have been soaked in distilled water for 48 hours.

### DETAILED DESCRIPTION

The present disclosure provides a method for stratifying subjects with respect to their relative risk of developing anaphylaxis upon the ingestion of a peanut allergen as well as formulations/reagents for performing such methods. The subjects can be mammals and, in some embodiments, humans. Broadly, the method of the present disclosure allows the stratification of subjects into two groups: a first group of subjects having an increased risk of developing anaphylaxis (e.g., high risk group) and a second group of subjects having a decreased risk of developing anaphylaxis (e.g., low risk group). The method is based on the subjects' differential ability to mount an allergic immune response against the peanut Ara h 2 allergen (for the high risk or anaphylaxis group, suspected of having IgE antibodies against the Ara h 2 allergen) or the Ara h 8 allergen (for the low risk or oral symptoms group, suspected of having IgE antibodies against the Ara h 8 allergen). Subjects which have been stratified in the high risk group can receive tailored recommendations and treatments. For example, subjects in the high risk group can receive a recommendation to perform oral immunotherapy to reduce their risk of developing anaphylaxis. Subjects which have been stratified in the low risk group can receive tailored recommendations and treatments.

Oral immunotherapy (OIT) is known in the art to refer to feeding an allergic individual an increasing amount of an allergen with the goal of increasing the threshold that triggers a reaction. In the context of the present disclosure, OIT is used to increase the amount of a peanut allergen (which may be, in some embodiments, a whole peanut or derived from a whole peanut) that triggers an allergic reaction (oral symptoms or anaphylaxis. For example, a person allergic to peanuts may be given very small amounts of peanut protein that would not trigger a reaction. This small amount is gradually increased over a period of months. The goal of therapy is to raise the threshold that may trigger a reaction and provide the allergic individual protection against accidental ingestion of the allergen. OIT is not necessarily a curative therapy and can be used to desensitize or reduce the risk of anaphylaxis.

The present disclosure also provides a peanut hypoallergenic formulation that can be used, for example, in the stratification methods and as well as during oral immunotherapy. As used in the context of the present disclosure, a “peanut hypoallergenic formulation” refer to a composition which is less allergenic than a whole peanut (which can, in some embodiments, be raw or boiled) but can nevertheless induce an allergic reaction at least in subjects having already experienced a first anaphylactic shock upon the ingestion of a peanut allergen.

It is known in the art that whole peanuts include at least the following protein-derived allergens: Ara h 1, Ara h 2, Ara h 3, Ara h 4, Ara h 5, Ara h 6, Ara h 7, Ara h 8, Ara h 9, Ara h 10, Ara h 11, Ara h 12, Ara h 13, Ara h 14, Ara h 15, Ara h 16, Ara h 17 and Ara h 18. The “peanut hypoallergenic formulation” and the “first reagent” of the present disclosure comprises the Ara h 2 allergen but substantially lack (or are devoid of) the Ara h 8 allergen. As used in the context of the present disclosure, the expression “substantially lack the Ara h 8 allergen” indicates that the peanut hypoallergenic formulation or the first reagent have less than 10%, 9%, 8%, 7%, 6%, 5%, 4%, 3%, 2%, 1%, 0.9%, 0.8%, 0.7%, 0.6%, 0.5%, 0.4%, 0.3%, 0.2%, 0.1% or less of the amount of the Ara h 8 allergen that is present in the whole peanut. In some embodiments, the peanut hypoallergenic formulation or the first reagent does not have a detectable amount of the Ara h 8 allergen. In some embodiments, the peanut hypoallergenic formulation or the first reagent is devoid of the Ara h 8 allergen. In some embodiments, the peanut hypoallergenic formulation or the first reagent have a reduced amount of the Ara h 2 allergen when compared to the whole peanut. However, the peanut hypoallergenic formulation has enough Ara h 2 allergen so as to elicit an allergic immune response in subjects belonging to the high risk group. The peanut allergenic formulation and the first reagent of the present disclosure can have one or more additional Ara h 1, Ara h 3, Ara h 4, Ara h 5, Ara h 6, Ara h 7, Ara h 8, Ara h 9, Ara h 10, Ara h 11, Ara h 12, Ara h 13, Ara h 14, Ara h 15, Ara h 16, Ara h 17 or Ara h 18 allergen, in the same or a different amount when compared to the whole peanut. The peanut allergenic formulation and the first reagent of the present disclosure can include Ara h 1 allergen, in the same or a different amount when compared to the whole peanut. The peanut allergenic formulation and the first reagent of the present disclosure can include Ara h 2 allergen, in the same or a different amount when compared to the whole peanut. The peanut allergenic formulation and the first reagent of the present disclosure can include Ara h 3 allergen, in the same or a different amount when compared to the whole peanut. The peanut allergenic formulation and the first reagent of the present disclosure can include Ara h 4 allergen, in the same or a different amount when compared to the whole peanut. The peanut allergenic formulation and the first reagent of the present disclosure can include Ara h 5 allergen, in the same or a different amount when compared to the whole peanut. The peanut allergenic formulation and the first reagent of the present disclosure can include Ara h 6 allergen, in the same or a different amount when compared to the whole peanut. The peanut allergenic formulation and the first reagent of the present disclosure can include Ara h 7 allergen, in the same or a different amount when compared to the whole peanut. The peanut allergenic formulation and the first reagent of the present disclosure can include Ara h 9 allergen, in the same or a different amount when compared to the whole peanut. The peanut allergenic formulation and the first reagent of the present disclosure can include Ara h 10 allergen, in the same or a different amount when compared to the whole peanut. The peanut allergenic formulation and the first reagent of the present disclosure can include Ara h 11 allergen, in the same or a different amount when compared to the whole peanut. The peanut allergenic formulation and the first reagent of the present disclosure can include Ara h 12 allergen, in the same or a different amount when compared to the whole peanut. The peanut allergenic formulation and the first reagent of the present disclosure can include Ara h 13 allergen, in the same or a different amount when compared to the whole peanut. The peanut allergenic formulation and the first reagent of the present disclosure can include Ara h 14 allergen, in the same or a different amount when compared to the whole peanut. The peanut allergenic formulation and the first reagent of the present disclosure can include Ara h 15 allergen, in the same or a different amount when compared to the whole peanut. The peanut allergenic formulation and the first reagent of the present disclosure can include Ara h 16 allergen, in the same or a different amount when compared to the whole peanut. The peanut allergenic formulation and the first reagent of the present disclosure can include Ara h 17 allergen, in the same or a different amount when compared to the whole peanut. The peanut allergenic formulation and the first reagent of the present disclosure can include Ara h 18 allergen, in the same or a different amount when compared to the whole peanut. The peanut hypoallergenic formulation and the first reagent of the present disclosure can be derived from whole peanuts.

The present disclosure also provides a process for making a peanut hypoallergenic formulation. The process comprises providing an initial composition comprising both the Ara h 2 and the Ara h 8 allergen. The initial composition can comprise or be derived from a whole peanut. The initial composition can comprise or be derived from a whole peanut which has been previously submitted to a thermal treatment such as, for example, a roasting step, a frying step and/or a boiling step. As such, in some embodiments, the process can include a preliminary heat treatment step to provide the initial composition (such as cooking, roasting, frying or boiling the initial composition). In some embodiments, the initial composition can be convert in a paste or butter form by submitting it, for example, to a pureeing or an extruding step (to provide the initial composition in a puree or extruded form). In some embodiments, the initial composition can be provided in a flour form and the process can include making a flour from the initial composition. In alternative embodiments, the initial composition comprises or is derived from a raw peanut. As such, in some embodiments, the process can exclude a preliminary heat treatment step. In some embodiments, the initial composition was not previously submitted to a previous pressure treatment step. In some embodiments, the initial composition is not derived from a whole peanut which has been previously submitted to a simultaneous heat and pressure treatment (e.g., autoclaved peanuts).

In the process of the present disclosure, the initial composition is submitted to a simultaneous heat treatment and pressure treatment step, e.g., the initial composition is heated and submitted to a pressure at the same time. In some embodiments, the process includes heating the initial composition to a minimal temperature of at least 120° C., 121° C., 122° C., 123° C., 124° C., 125° C., 126° C., 127° C., 128° C., 129° C., 130° C., 131° C., 132° C., 133° C., 134° C., 135° C., 136° C., 137° C., 138° C., 139° C., 140° C. or higher. In some embodiments, the process includes heating the initial composition to a maximal temperature of at most 140° C., 139° C., 138° C., 137° C., 136° C., 135° C., 134° C., 133° C., 132° C., 131° C., 130° C., 129° C., 128° C., 127° C., 126° C., 125° C., 124° C., 123° C., 122° C., 121° C. or lower. In still a further embodiment, the process includes heating the initial composition to a temperature between 120° C. and 140° C. In some embodiments, the process includes submitting the initial composition to a minimal pressure of 1.15, 1.20, 1.25, 1.30, 1.35, 1.40, 1.45, 1.50, 1.55, 1.60, 1.65, 1.70, 1.75, 1.80, 1.85, 1.90, 1.95, 2.00, 2.05, 2.10, 2.15, 2.20, 2.25, 2.30, 2.35, 2.40, 2.45, 2.50, 2.55, 2.60 atm or higher. In some embodiments, the process includes submitting the initial composition to a maximal pressure of 2.60, 2.55, 2.50, 2.45, 2.40, 2.35, 2.30, 2.25, 2.20, 2.15, 2.10, 2.05, 2.00, 1.95, 1.90, 1.85, 1.80, 1.75, 1.70, 1.65, 1.60, 1.55, 1.50, 1.45, 1.40, 1.35, 1.30, 1.25, 1.20, 1.15 atm or lower. The pressure treatment can include, in some embodiments, applying a vapor (e.g., water) pressure to the initial composition. In some embodiments, the simultaneous heat and pressure treatment can be applied for a minimal period of 2, 3, 4, 5, 6, 7, 8, 9, 10, 11, 12, 13, 14, 15, 16, 17, 18, 19, 20, 21, 22, 23, 24, 25, 26, 27, 28, 29, 30 minutes or more. In some embodiments, the simultaneous heat and pressure treatment can be applied fora maximal period of 30, 29, 28, 27, 26, 25, 24, 23, 22, 21, 20, 19, 18, 17, 16, 15, 14, 13, 12, 11, 10, 9, 8, 7, 6, 5, 4, 3, 2 minutes or less. In some specific embodiments, the simultaneous heat and pressure treatment can be applied for a period of times between 2 and 30 minutes. In some embodiments, the simultaneous heat and pressure treatment can be applied by autoclaving the initial composition. The person skilled in the art will recognized that the various parameters of the simultaneous heat and pressure treatment can be modified to obtain the peanut hypoallergenic reaction.

In some embodiments, once the peanut hypoallergenic formulation has been obtained it can be used directly in the methods described herein without further modifications. In some embodiments, the protein/peptide content peanut hypoallergenic composition is not further modified prior to being used in methods and therapies. In some embodiments, the peanut hypoallergenic composition can be further modified prior to being used. For example, the process can include, in some embodiments, cooking the peanut hypoallergenic formulation (for example by frying, roasting or boiling the peanut hypoallergenic formulation to provide it in a cooked form (e.g., a fried, roasted or boiled form). In another example, the process can include converting the peanut hypoallergenic formulation into a paste or butter form (for example by pureeing or extruding it to provide it in a puree or extruded form). In an additional example, the process can include adding one or more further components to the formulation such as, for example, a color, a flavor, a taste modifier, a preservative, a buffer, etc.

In still a further example, the process can include formulating the peanut hypoallergenic formulation in a skin prick reagent. This can be achieved, for example, by making a paste of the peanut hypoallergenic formulation, removing the fat from the paste to obtain a peanut hypoallergenic flour and dissolving the peanut hypoallergenic flour in a physiologically acceptable diluent (in some embodiments a buffer). The defatting or fat removing step can be completed by dissolving the peanut paste in an alcohol and removing the alcohol to provide the peanut hypoallergenic flour.

In yet another example, the process can include formulating the peanut hypoallergenic formulation in an oral immunotherapy agent. This can be achieved, for example, by drying the peanut hypoallergenic formulation and grinding it into a powder. In some embodiments, the peanut hypoallergenic powder is encapsulated with a filer.

In still another example, the process can include formulating the peanut hypoallergenic formulation in a food product. This can be achieved, for example, by combining the peanut hypoallergenic formulation with one or more other ingredients (a carbohydrate source, a protein source and/or a lipid source) to make it more palatable to the subject (which can be a mammal, such as a human).

In some embodiments, the peanut hypoallergenic formulation has a ¹H nuclear magnetic resonance CH NMR) spectrum profile at least substantially similar or identical to the ¹H NMR spectrum profile shown in FIG. 10C, in the ranges between 0.5 and 2 ppm and between 6.5 and 8.5 ppm. The ranges between 0.5 and 2 ppm and between 6.5 and 8.5 ppm of ¹H NMR spectrum profile correspond to the proteins, protein fragments and peptides that are present in the peanut hypoallergenic formulation. In some embodiments, the ¹H NMR spectrum profile can be obtained by performing a solution ¹H NMR (e.g., on the water soluble components of the peanut hypoallergenic formulation) or high-resolution magic angle spinning (HR-MAS)¹H NMR.

The process can include determining the presence, amount or the level of degradation of Ara h 2 and/or Ara h 8 allergen in the peanut hypoallergenic formulation. This can be done for example to make sure that the simultaneous heat and pressure treatment which has been applied substantially reduced or degraded the Ara h 8 allergen, while maintaining acceptable amounts of the Ara h 2 allergen. This can be achieved, for example, by performing a ¹H NMR analysis on the peanut hypoallergenic formulation. In some embodiments, the process can include performing a solution ¹H NMR or high-resolution magic angle spinning (HR-MAS)¹H NMR on the peanut hypoallergenic formulation. In some additional embodiments, the process can include performing a solution ¹H NMR or high-resolution magic angle spinning (HR-MAS)¹H NMR on the initial composition for comparison. In some embodiments, the ¹H NMR spectrum of the peanut hypoallergenic formulation or the initial composition can be compared to the ¹H NMR spectrum of FIG. 10C to determine if the simultaneous heat and pressure treatment which has been applied substantially reduced or degraded the Ara h 8 allergen, while maintaining acceptable amounts of the Ara h 2 allergen. The comparison can include comparing the location of at least one or a plurality of peaks and/or the intensity of at least one or a plurality of peaks. The comparison is preferentially performed in the in the range between 0.5 and 2 ppm and between 6.5 and 8.5 ppm which is associated to the proteins, protein fragments and peptides of the formulation or composition. The expression “a ¹H nuclear magnetic resonance CH NMR) spectrum profile at least substantially similar to the ¹H NMR spectrum profile shown in FIG. 10C” refers to the fact that the solution ¹H NMR spectrum profile of the peanut hypoallergenic formulation has the majority of the peaks (more than 50, 55, 60, 65, 70, 75, 80, 85, 90, 95, 96, 97, 98 or 99%) present in the ¹H NMR spectrum profile with similar peak shaping, intensity and area as shown in FIG. 10C. The expression “a ¹H nuclear magnetic resonance CH NMR) spectrum profile is identical to the ¹H NMR spectrum profile shown in FIG. 10C” refers to the fact that the solution ¹H NMR spectrum profile of the peanut hypoallergenic formulation comprises all the peaks present in the ¹H NMR spectrum profile with identical shape, intensity and area ratio (relative to equal values of a TSP standard) as shown in FIG. 10C.

The present disclosure also provides a peanut hypoallergenic formulation. In some embodiments, the peanut hypoallergenic formulation has a ¹H nuclear magnetic resonance CH NMR) spectrum profile at least substantially similar or identical to the ¹H NMR spectrum profile shown in FIG. 10C in the range between 0.5 and 2 ppm and between 6.5 and 8.5 ppm. In yet additional embodiments, the peanut hypoallergenic formulation is obtained by the process described herein.

In some embodiments, the peanut hypoallergenic formulation can include adding one or more further components to the formulation such as, for example, a color, a flavor, a taste modifier, a preservative, a buffer, etc.

The present disclosure also provides a skin prick test reagent comprising the peanut hypoallergenic formulation described herein or obtained by the process described herein. In some embodiments, the skin prick test reagent can be provided in a resuspended form in a buffer (a Tris buffer for example). The skin prick test reagent can be obtained by making a paste or a butter of the peanut hypoallergenic formulation, defatting the paste or the butter to obtain a flour of the peanut hypoallergenic formulation and dissolving the flour in a solution (such as a buffer). The skin prick test reagent can be stored in a container (such as a vial). Alternatively, the skin prick test reagent can be provided in a flour form ready for resuspension. The skin prick test reagent can be stored at refrigeration temperature (4° C.) before being used in a skin prick test with a subject.

The present disclosure provides an oral immunotherapy reagent comprising the peanut hypoallergenic formulation described herein or obtained by the process described herein. The oral immunotherapy reagent of the present disclosure is especially useful in subjects having been stratified in the high risk group. In some embodiments, the oral immunotherapy reagent can be provided in a dried powder form. The oral immunotherapy reagent can be obtained by making a paste or a butter of the peanut hypoallergenic formulation, defatting the pasted or the butter to obtain a flour of the peanut hypoallergenic formulation. Alternatively, the peanut hypoallergenic formulation can be dried and ground into an oral immunotherapy agent. The oral immunotherapy reagent can be stored in a container (such as a vial). The oral immunotherapy reagent can be stored at refrigeration temperature (4° C.) or room temperature (20-30° C.) before being used in an oral immunotherapy with a subject.

The present disclosure provides a food product comprising the peanut hypoallergenic formulation described herein or obtained by the process described herein. The food product can be obtained by combining the peanut hypoallergenic formulation with at least one additional ingredient, which may be, for example, a carbohydrate, a lipid and/or a protein. The food product can also be processed prior to being used. For example, the food product can be pureed or extruded to provide an hypoallergenic peanut butter. In some embodiments, the hypoallergenic peanut butter could be consumed in subjects having been stratified in the low risk group.

The present disclosure also provide a kit for performing a skin prick test. The kit comprises a first reagent comprising a Ara h 2 allergen and substantially lacking or devoid of the Ara h 8 allergen. For example, the first reagent can be a substantially purified form of the Ara h 2 allergen. In still a further embodiment, the first reagent can be the peanut hypoallergenic formulation described herein. In another embodiment, the first reagent can be the skin prick test reagent described herein. The first reagent can be formulated for contacting a skin abrasion on the subject being submitted to the skin prick test.

The kit also comprises instructions to use the first reagent in a skin prick test. For example, the instructions can indicate that the skin of the subject which is being submitted to the skin prick test should be scratched or pricked to cause a skin abrasion. The instructions can also indicate that the first reagent should be contacted with the skin abrasion so as to allow the mounting of an allergic immune reaction at the site of contact. The instructions can also indicate to remove the first reagent. The instructions can, in some embodiments, indicate to measure the wheal and flare response around the site of contact. As used in the context of the present disclosure, a wheal and flare response refers to a swelling produced by the release of serum in the skin (wheal) and redness resulting from the dilation of blood vessels (flare). The presence or absence of the wheal and flare response can be determined by measuring the size of the inflamed and red area of the skin (usually the diameter of such area) that can be induced at the contact of an allergen. The instructions can also indicate that the presence of a wheal and flare response at the site of contact is indicative that the subject has an increased risk of developing anaphylaxis (upon the ingestion of a peanut allergen, such as a whole peanut) than a first control subject who do not exhibit a wheal and flare response with the first reagent around the site of contact.

In some embodiments, the kit can also comprise a second reagent comprising a Ara h 8 allergen. The second reagent can be a substantially purified form of the Ara h 8 allergen, comprise a peanut or be derived from a peanut which has not been submitted to a simultaneous heat and pressure treatment as described herein. The second reagent can be, for example, a whole peanut protein extract which may be derived from a whole peanut flour (which may be from a raw or roasted peanut) which may have been dissolved in a solution (such as a buffer).

The kit can also comprise instructions to use the second reagent in a skin prick test. For example, the instructions can indicate that the skin of the subject which is being submitted to the skin prick test should be scratched or pricked to cause a further (second) skin abrasion. The instructions can also indicate that the second reagent should be contacted with the second skin abrasion so as to allow the mounting of an allergic immune reaction at the site of contact. The instructions can also indicate to remove the second reagent from the site of contact. The instructions can, in some embodiments, indicate to measure the wheal and flare response around the site of contact. The instructions can also indicate that the presence of a wheal and flare response at the site of contact of the second reagent and the absence of a wheal and flare response at the site of contact of the first reagent is indicative that the subject has a decreased risk of developing anaphylaxis (upon the ingestion of a peanut allergen, such as a whole peanut) than a second control subject who exhibited a wheal and flare response with the first reagent around the site of contact. The instructions can also indicate that the presence of a wheal and flare response at the sites of contact of the first and second reagent is indicative that the subject has an increased risk of developing anaphylaxis (upon the ingestion of a peanut allergen, such as a whole peanut) than the first control subject who did not exhibit a wheal and flare response with the first reagent around the site of contact.

The kit can, in some embodiments, further comprise the oral immunotherapy reagent described herein or other known oral immunotherapy reagents such as, for example, Palforzia™. In such embodiment, the kit can also include instructions to use the oral immunotherapy reagent described herein for oral immunotherapy. For example, the instructions can include to use the oral immunotherapy reagent in subject which exhibited a wheal and flare response to the first reagent, the second reagent or both. In some specific embodiments, the instructions can indicate that the subjects which exhibited a wheal and flare response to the first reagent would benefit from an OIT with the peanut hypoallergenic formulation described herein or an oral immunotherapy agent comprising same. In some specific embodiments, the instructions can indicate that the subjects which exhibited a wheal and flare response to the second reagent but not to the first reagent would not benefit from an OIT with the peanut hypoallergenic formulation described herein or an oral immunotherapy agent comprising same.

In some embodiments, the kit described herein can be used with a subject which has previously been diagnosed with a peanut allergy. This previous determination can be made using a skin prick test or a serological assay.

The present disclosure also provides a method for stratifying the risk of a subject to develop anaphylaxis upon the ingestion of a peanut allergen (which may be, for example, a whole peanut or a product derived therefrom). The stratification method can be used to tailor the treatment regimen of the stratified subjects and, in some embodiments, to adapt the oral immunotherapy to the stratified subjects. The method can be used to determine if a subject is suitable or not to receive a first dose of oral immunotherapy (e.g., start oral immunotherapy). The method can also be used to determine if a subject is suitable to continue to received a further dose of oral immunotherapy (e.g., continue oral immunotherapy). In some embodiments, the method can be used for screening subjects eligible for oral immunotherapy, remaining on the oral immunotherapy, and/or any form of therapy that relates to peanut allergy (such as, for example, the administration of nanocapsules to deliver peanut allergen, proteolysed peanut allergen, metronomics to deliver low doses of peanut allergens, etc.). The method relies on performing a skin prick test on a subject with the first reagent described herein (which may be provided with the kit described herein). As such, in a first step, the method comprises pricking or scratching the skin of the subject at a first location to make a first skin abrasion or providing a subject which already has a first skin abrasion at a first location. In a second step, the method comprises contacting the first reagent as described herein with the first skin abrasion at the first location. In some embodiments, the method can comprise removing the first reagent from the first skin abrasion after a certain period of time. In a third step, the method comprises determining the presence of an allergic reaction at the first location contacting with the first reagent. This can be done, for example, by determining the presence of a wheal and flare response at the first location. A subject exhibiting a wheal and flare response towards the first reagent at the first location is stratified as having an increased risk of developing anaphylaxis upon the ingestion of the peanut allergen. This increased risk is relative to a first control subject that did not exhibit an allergic reaction (e.g., a wheal and flare in response) to the first reagent. The first control subject can be a subject which is known not to have a peanut allergy or a subject which is allergic to peanuts but known to exhibit oral symptoms (e.g., subjects having IgE antibodies towards the Ara h 8 allergen for example).

The subject being stratified may or may not have been previously diagnosed with a peanut allergy. In embodiments in which the subject being stratified has previously been diagnosed with a peanut allergy, such subject may have previously been submitted to a skin prick test or a serological assay or both. The subject may or may not have experienced a previous anaphylaxis in response to a peanut allergen.

In some embodiments, the method can include using a second reagent as described herein (which can be provided as the kit provided herein) in the stratification method. This embodiment may be useful in subjects which did not exhibit an allergic response to the first reagent (but are nevertheless suspected of being allergic to a peanut allergen). In such embodiment, the method comprises pricking or scratching the skin of the subject at a second location to make a second skin abrasion or providing a subject which already has a second skin abrasion at a second location. The method comprises contacting the second reagent as described herein with the second skin abrasion at the second location. In some embodiments, the method can comprise removing the second reagent from the second skin abrasion after a certain period of time. The method comprises determining the presence of an allergic reaction at the second location contacted with the second reagent. This can be done, for example, by determining the presence of a wheal and flare response at the second location. A subject exhibiting a wheal and flare response at the second location but not at the first location is stratified as having a decreased risk of developing anaphylaxis upon the ingestion of the peanut allergen. This decreased risk is relative to a second control subject that did exhibit an allergic reaction (e.g., a wheal and flare in response) to the first reagent. The second control subject can be a subject which is known to have a peanut allergy and, in some embodiments, previously experienced anaphylaxis upon the ingestion of a peanut allergen.

In some embodiments, the method can allow for screening for subjects suitable for an oral immunotherapy. The risk of oral immunotherapy may be life threatening and, in some embodiments, oral immunotherapies should be administered in subjects having an increased risk of developing anaphylaxis. In some embodiments, the method provides suggesting or submitting the stratified subject to an oral immunotherapy. In such embodiments, it is possible to orally administer the oral immunotherapy agent or the food product to the stratified subject.

The present invention will be more readily understood by referring to the following examples which are given to illustrate the invention rather than to limit its scope.

### Example I—Characterization of Processed Peanut Compositions

Physical Peanut Processing. Commercially purchased peanuts (Montreal Food Store, Canada) were purchased raw and shelled. Peanuts were roasted with their seed coating in a convection oven at 150° C. for 30 minutes or were autoclaved in a tabletop autoclave at 136° C. at 2.48 Bar (36 psi) for 30 minutes. Additionally, roasted peanuts were autoclaved (Roast-Auto) and autoclaved peanuts were roasted (Auto-Roast), after a 30-minute pause at room temperature between processing methods. Analyses were performed in comparison with raw peanut (unprocessed).

Defatting into flour. Raw, roasted and autoclaved peanuts (6 to 12 of each) were ground into a smooth paste using a coffee grinder. The smooth paste was then suspended in hexanes and the peanut flour was collected by filtration under vacuum.

Preparation of Whole Peanut Protein Extracts. Dry peanut flours (raw, roasted and autoclaved) were processed into whole peanut protein extracts using a 20 mM Tris Buffer (pH 8.5), following the protocol optimized by Walczyk et al. (2017). Protein extract concentrations were determined by Bradford Assay using bovine serum albumin (BSA) as a standard. Concentrations were then adjusted to equal values across various processing conditions accordingly depending on the experiment being performed.

Electrophoresis. Whole peanut protein extracts were adjusted to equal concentrations of 1 mg/mL and were analyzed using sodium dodecyl sulfate-polyacrylamide gel electrophoresis (SDS-PAGE) under reducing conditions (2.5% β-mercaptoethanol).

Western Blot Analysis. Following electrophoresis, proteins were transferred to PVDF membranes for 150 min at 100 V. Membranes were washed in TBST and then blocked with 5% BSA for 1 hour. Rabbit polyclonal IgG anti-Ara h 1, 2 and 8 antibodies (Indoor Biotechnologies, VA, USA) diluted in the same buffer were incubated overnight at 4° C. Membranes were then washed and a donkey anti-rabbit IgG monoclonal antibody conjugated with horseradish peroxidase (HRP) was incubated for 1 hour at room temperature. After several washings, the membranes were incubated with the Clarity and Clarity Max enhanced chemiluminescence (ECL) substrates (Bio-Rad Laboratories, Canada) and then images were taken with the ChemiDoc XRS+ Imaging System.

Determination of Specific IgE Responses to Whole Peanut Extracts. The IgE-binding capacity of the proteins of the various processing conditions was analyzed using the enzyme-linked immunosorbent assay (ELISA). 96-well polystyrene plates were coated with the different protein extracts (raw, roasted, autoclaved) at a concentration of 10 μg/mL overnight at 4° C. Plates were then washed with 0.1% Tween 20 in PBS 1× and were then blocked with 100 μL per well of 1% BSA (blocking buffer) for 1 hour at room temperature. The serum of each of 4 patients highly allergic to peanuts was diluted in the same buffer at 1:1000 and 50 μL per well was incubated for 2 hours at room temperature. A serial dilution of recombinant human IgE antibody at 50 ng/ml (ELISA Ready-SET-Go! Kit, Thermo Fisher Scientific, ON, Canada) binding to equal concentrations of a capture antibody bound to the plate was used to construct a standard curve by plotting the known concentrations of the standard values versus the optical density (OD) at 450 nm.

Relative quantification of Ara h 1, 2 and 8 in Whole Peanut Extracts. The same protocol as the peanut-specific ELISA was used. However, the primary antibody in this case was the individual polyclonal IgG anti-Ara h 1, 2 and 8 antibodies (Indoor Biotechnologies, VA, USA) used in the Western blot experiments. In the case of anti-Ara h 1 and 2, the plate was coated with a concentration of 0.1 μg/mL of peanut protein, and 10 μg/mL in that of anti-Ara h 8. The primary antibody was diluted in blocking buffer at 1:1000 and 50 μL per well was incubated for 2 hours at room temperature.

Solution ¹H NMR Spectroscopy. Six whole peanuts from each condition were placed in 10 mL of double distilled water in a 15 mL falcon tube. The peanuts were allowed to soak in the water at room temperature for 48 hours. Three samples per condition (1 mL each) were evaporated under a SpeedVac at 45° C. for 1.5 hours and the resulting residue reconstituted in 0.6 mL of double distilled water. The three samples were combined together to give a total volume of 1.8 mL, 450 μl of which was collected for analysis.

Solution ¹H NMR spectra were run on a Bruker 400 MHz NMR spectrometer for analysis using the water suppression pulse sequence, zgpr (Bruker standard sequence). 32 scans were acquired with an acquisition time of 3 s and a spectral width of 12 kHz. The ¹H chemical shifts were internally referenced by adding 0.5 mM of deuterated 3-(trimethylsilyl)propionic-2,2,3,3,-d₄ acid sodium salt (TSP-d₄) set to 0.0 ppm.

Skin Prick Testing. Peanut-allergic subjects and non-allergic healthy controls aged 4 to 30 years old were recruited to the Montreal Children's Hospital for a Skin Prick Testing (SPT) after obtaining informed consent. Subjects were considered allergic based on previous history suggestive of immediate allergy to peanut and the presence of previous positive SPT to peanut (wheal diameter greater than 7 mm), detection of serum-specific IgE to peanut proteins (>0.35 kU/L) or a positive oral food challenge test to peanut.

In a first pilot SPT study, peanuts processed by roasting, autoclaving, roasting then autoclaving, and autoclaving then roasted, along with raw peanuts, were processed into protein extracts as described above. Extracts of each condition were diluted to equal concentrations equivalent to the standard peanut extract (Allergy Canada Limited, Thornhill, ON, LOT No: 3467710) as measured by Bradford Assay. The SPTs were conducted by placing a drop of each peanut protein extract, as well as the standard commercial extract on the forearm and making a small scratch on the arm using a solid bore needle. Saline diluent and Histamine (1 mg/mL, ALK-Abello Pharm., Inc., Mississauga, ON) were used as negative and positive controls, respectively. After 10 minutes, the size of the wheal diameter was measured (in mm), which correlates with the presence of peanut-specific IgE. All skin tests were performed by an experienced allergy nurse. Research Ethics Board (REB) approval was obtained from the McGill University Health Centre (MUHC)-REB (2020-5745).

A second SPT study was conducted in patients with suspected peanut allergy using the current, standard, whole peanut protein extract, as well as an extract created from autoclaved peanuts. Patients who received a positive SPT result (wheal diameter greater than 3 mm) to either extract were invited to provide a blood sample for total peanut- and component-specific serum IgE analysis via ELISA as described above. Research Ethics Board (REB) approval was obtained from the McGill University Health Centre (MUHC-REB approval: 2021-7628).

Statistical Analysis. All results were statistically analyzed using GraphPad Prism Version 5.00 (GraphPad Software, San Diego, Calif., USA). Analysis of Variance (ANOVA) was used to determine significant differences between normalized IgE binding values of each processing condition. A two-tailed, paired Student's t-test was used to determine significant differences between wheal diameters using raw and autoclaved peanut protein extracts. The level of significance was set at p<0.05 in both cases.

Peanuts of each condition (raw, roasted and autoclaved) were defatted and processed into a protein extract using a 20 mM Tris buffer (pH 8.5). The relative protein concentrations were quantified using a Bradford assay normalized to the raw peanut extract and are presented in Table 1. The roasted and autoclaved extracts yielded approximately half and 4 to 10%, respectively, when compared to the raw protein extract concentration. For all experiments, protein concentrations were adjusted to equal values.

TABLE 1
Mean concentration of whole protein extracts of each condition
following a Bradford Assay.

| | Mean Concentration ± SD |
|---|---|
| Condition | (normalized to raw), n = 6 |
| Raw | 100.0% |
| Roast | 45.6% ± 19.7% |
| Autoclave | 8.0% ± 1.9% |
| Autoclave-Roast | 4.2% ± 1.0% |
| Roast-Autoclave | 7.0% ± 2.6% |

Following gel electrophoresis and transfer to PVDF membranes, Western blots were performed on peanut protein extracts of the conditions described above using antibodies specific for Ara h 1, 2 and 8.

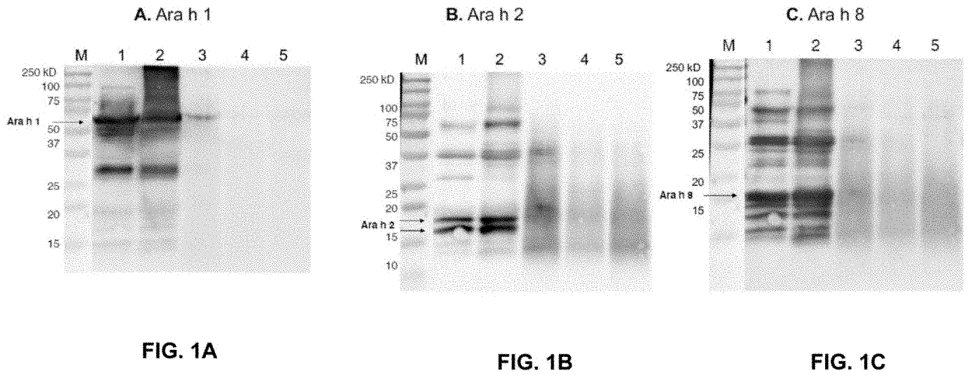

FIG. 1A shows a Western blot using an antibody selective for Ara h 1, a 64-kilodalton (kDa) 7S globulin. Both the raw and roasted peanut extracts appear to have the highest proportion of Ara h 1 as demonstrated by the greatest band intensity. The autoclaved peanut protein extracts have very little to no detection of Ara h 1 via Western blot.

Ara h 2 is a 2S albumin protein of size 17 kDa and has been established as the most potent peanut allergen, being recognized by the serum IgE of over 90% of peanut-allergic patients. Moreover, recent literature suggests that Ara h 2 exists as two distinct isoforms in mature peanuts, differing by a stretch of 12 amino acids, and that the larger of the two isoforms may be a more potent allergen. In FIG. 1B, the presence of two distinct and intense bands were observed in the roasted extract and to a lesser degree in the raw extract. In all autoclaved extracts, no distinct bands were observed for Ara h 2 but a general smear throughout the lanes.

In the case of Ara h 8, a plant panallergen of size 17 kDa, we observed a similarly high band intensity for both the raw and roasted peanut extracts, and very little detection in the autoclaved extracts (FIG. 1C).

Roasting peanut either before or after autoclaving (lanes 5 and 4, respectively) did not alter the levels of Ara h 1, 2 or 8 detection.

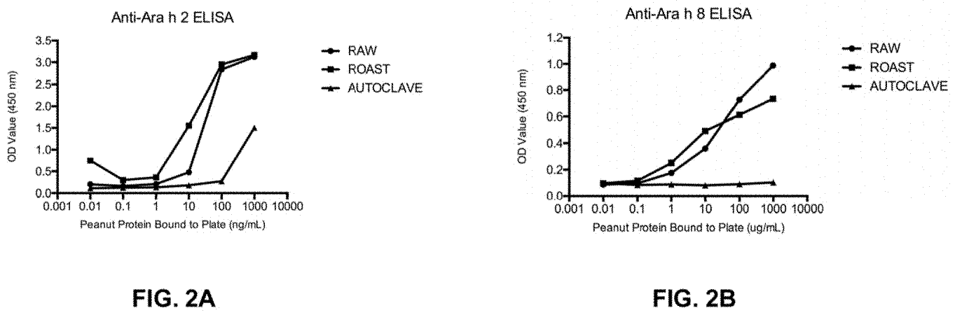

In order to determine whether the autoclaving process denatured specific allergenic proteins, an enzyme-linked immunosorbent assay (ELISA) was performed to quantify the relative amounts of Ara h 2 and 8 present in the whole peanut protein extracts (FIG. 2). In the case of Ara h 2, raw and roasted protein extracts showed similarly high levels of detection at a protein concentration of 1 μg/mL coated on the plate. In the case of the autoclaved extract, detection was reduced by approximately 50% for the same concentration (FIG. 2A).

FIG. 2B shows similar detection levels of Ara h 8 for raw and roasted peanut proteins at high extract concentrations (1 mg/mL). Interestingly, the autoclaved peanut extracts detected no Ara h 8, independent of extract concentration.

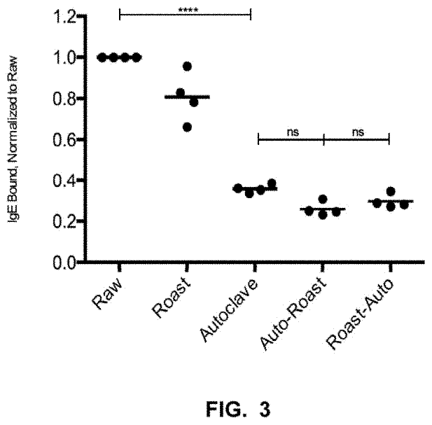

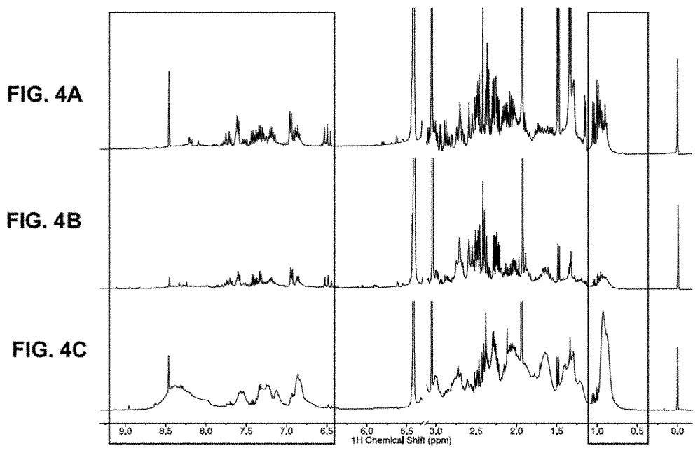

With the aim of understanding what effect autoclaving has on the peanut allergens in the context of IgE binding, peanut-specific IgE binding was quantified using the ELISA assay with the serum from highly allergic patients as the primary antibody. IgE binding decreased significantly upon peanut autoclaving (FIG. 3, p<0.0001). This result was the same when peanuts were roasted either before or after autoclaving. No significant change in IgE binding was observed when comparing the roasted peanut to raw.

In order to obtain a snapshot of their small molecule profiles, peanuts of each condition were soaked in distilled water for 48 hours and the resulting solution was analyzed by ¹H NMR spectroscopy (FIG. 4). This technique allows for rapid analysis with minimal sample preparation and provides a characteristic spectrum dependent on the chemical environments of individual ¹H nuclei. The spectra of the raw and roasted peanut-soaked solutions do not differ greatly in peak distribution or intensity (FIGS. 4A & B). However, the spectrum of the autoclaved peanut-soaked solution differed considerably. Particularly, in the regions between 0.5 ppm and 2.0 ppm as well as between 6.5 ppm and 8.5 ppm, the autoclaved peanut-soaked spectrum has a different peak distribution with a series of broad peaks (FIG. 4C), which is not seen in that of the raw or roasted spectra.

In an attempt to assess IgE binding in vivo, peanut-allergic and non-allergic healthy controls were skin prick tested (SPT) with the panel of protein extracts created from raw, roasted and autoclaved peanuts and the resulting wheal diameters were measured (Table 2). Within the peanut-allergic group, a statistically significant reduction in mean wheal diameter was observed in the autoclaved extract when compared to the raw extract (p<0.05).

TABLE 2
Skin Prick Test (SPT) results displaying the mean wheal
diameter in millimetres.

| | Mean Wheal Diameter^{‡} (mm) | | | |
|---|---|---|---|---|
| | Allergic^{†} (n = 9) | | | |
| | | Severely | Oral | Non- |
| | | Allergic | Symptoms | Allergic |
| Extract Used | | (n = 5) | (n = 4) | (n = 3) |
| Standard | 10.2 | 12.8 | 7.0 | 1.3 |
| Raw | 9.6* | 12.4 | 6.0 | 1.0 |
| Roast | 9.9 | 9.0 | 11.0 | 1.3 |
| Autoclave | 6.9** | 11.0 | 1.8* | 1.0 |
| Autoclave-Roasted | 6.7 | 10.2 | 2.3 | 1.3 |
| Roasted-Autoclave | 6.9 | 10.6 | 2.3 | 1.0 |
| (+) Control (Histamine) | 4.2 | 4.2 | 4.3 | 4.3 |
| (−) Control (Saline) | 0.7 | 0.6 | 0.8 | 1.3 |

*p < 0.05,
**p < 0.01, Student's t-test compared to raw.
^{‡}Results under 3 mm are clinically insignificant.
^{†}Allergic subjects were divided into two sub-groups based on previous exposure to peanut: those at risk for anaphylaxis and those who experience only oral symptoms.

Additional information was revealed when the allergic group was further stratified into two groups of ‘highly’ and ‘mildly’ allergic patients. Those patients in the ‘highly’ allergic group have previously experienced a severe allergic reaction to peanut or have demonstrated high likelihood of severe allergy based on laboratory tests (SPT wheal size or serum IgE levels). Those in the ‘mildly’ allergic group previously experienced only oral symptoms upon peanut consumption.

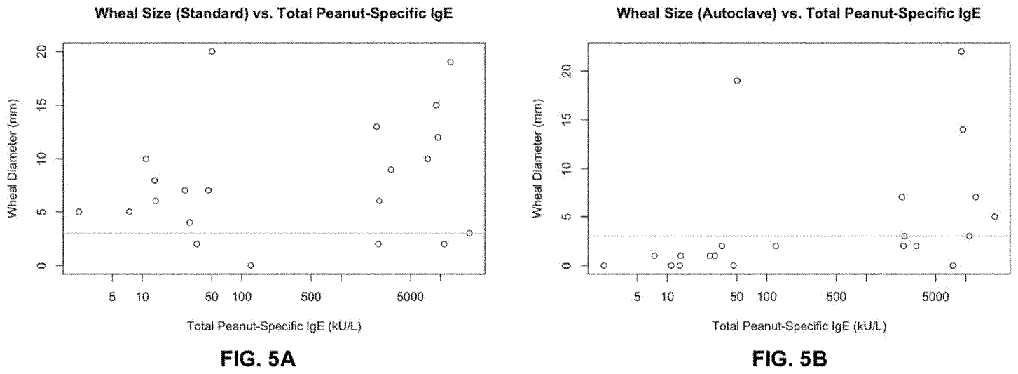

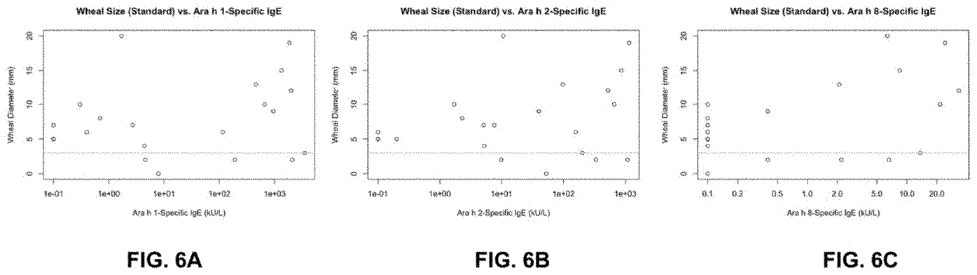

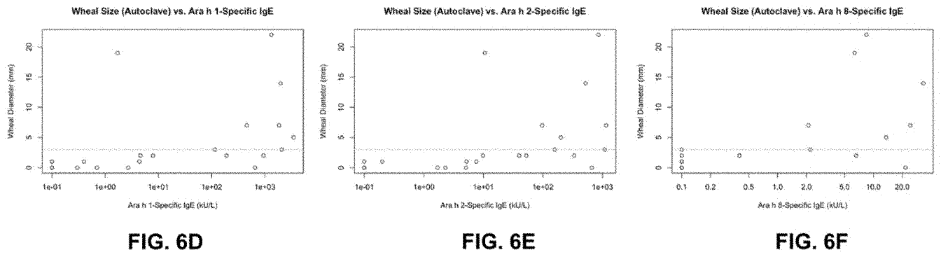

This was further confirmed upon a larger-scale follow-up SPT study using only the current, standard peanut extract as well as an extract created from autoclaved peanuts (Table 3). The total peanut-specific IgE (sIgE) and component-sIgE were quantified for patients that provided blood samples via ELISA. Interestingly, two distinct groups of patients based on their total-sIgE levels were observed, where the group with high sIgE levels experience a positive SPT result to both extracts, while the low-sIgE group receive a positive SPT to the standard extract (wheal diameter greater than 3 mm), but a negative SPT to the autoclaved peanut extract (FIG. 5). These trends continued with the component-sIgE analysis, where IgE levels specific for protein allergens Ara h 1, Ara h 2 and Ara h 8 were quantified (FIG. 6).

TABLE 3
Patient experimental data. Wheal diameters (WD) measured by SPT displayed in millimeters (mm)
using commercial standard and autoclaved peanut extracts. Total peanut (PN)-, Ara h 1-, Ara h 2-,
and Ara h 8-specific IgE (sIgE) levels as measured by in vitro ELISA displayed in kU/L.

| Study | Standard | Autoclave | Total PN- | Ara h 1- | Ara h 2- | Ara h 8- |
|---|---|---|---|---|---|---|
| Subject | WD (mm) | WD (mm) | sIgE (kU/L) | sIgE (kU/L) | sIgE (kU/L) | sIgE (kU/L) |
| 01 | 6 | 3 | 2417.4 | 115.2 | 158.0 | 0.0 |
| 02 | 7 | 1 | 26.8 | 0.1 | 7.6 | 0.0 |
| 03 | 4 | 1 | 30.0 | 4.4 | 5.2 | 0.0 |
| 04 | 7 | 0 | 46.2 | 2.7 | 5.1 | 0.0 |
| 05 | 15 | 22 | 9078.0 | 1328.2 | 858.5 | 8.5 |
| 06 | 13 | 7 | 2281.4 | 457.1 | 97.6 | 2.1 |
| 08 | 2 | 2 | 35.3 | 4.6 | 9.8 | 6.6 |
| 09 | 20 | 19 | 50.2 | 1.7 | 10.6 | 6.4 |
| 10 | 19 | 7 | 12641.6 | 1856.6 | 1149.7 | 24.3 |
| 11 | 10 | 0 | 10.9 | 0.3 | 1.7 | 0.0 |
| 12 | 2 | 3 | 10898.0 | 2091.4 | 1087.4 | 2.2 |
| 13 | 12 | 14 | 9431.3 | 1982.7 | 522.5 | 33.3 |
| 14 | 6 | 1 | 13.6 | 0.4 | 0.1 | 0.1 |
| 15 | 5 | 1 | 7.4 | 0.0 | 0.2 | 0.0 |
| 16 | 2 | 2 | 2371.4 | 190.2 | 333.6 | 0.4 |
| 17 | 3 | 5 | 19547.9 | 3471.6 | 201.5 | 13.6 |
| 18 | 8 | 0 | 13.3 | 0.7 | 2.3 | 0.0 |
| 19 | 9 | 2 | 3180.0 | 944.2 | 40.2 | 0.4 |
| 20 | 0 | 2 | 123.1 | 7.9 | 53.0 | 0.0 |
| 21 | 5 | 0 | 0.0 | 0.0 | 0.0 | 0.0 |
| 22 | 10 | 0 | 7442.8 | 656.2 | 661.3 | 21.6 |
| 23 | 5 | 0 | 0.0 | 0.0 | 0.0 | 0.0 |
| 24 | 5 | 0 | 2.3 | 0.0 | 0.0 | 0.0 |

The results show similar wheal sizes when comparing the standard, commercial peanut extract to that of the raw peanut created in the lab. However, a striking decrease in wheal size is observed when using autoclaved extracts in the mildly allergic group. In contrast, this was not observed in the case of the highly allergic group. The roasted peanut extract showed a slight decrease in wheal size in the highly allergic group and an increase in wheal size in the mildly allergic group.

In this example, the effects of roasting, autoclaving and their interaction on the major peanut protein allergens and thus, peanut allergenicity were characterized. It was demonstrated that high-pressure and temperature autoclaving reduces the detection of the major allergens Ara h 1, Ara h 2, and Ara h 8 as shown by Western blot and ELISA (FIGS. 1 & 2) as well as peanut-specific IgE binding in vitro (FIG. 3) when compared to raw or roasted peanuts. Moreover, the effect of autoclaving dominates over roasting in all experiments performed as shown by roasting the peanuts either before or after autoclaving.

In all experiments performed in this example, the results did not differ significantly between the raw and roasted peanut conditions. This is contrast to previously reported findings: Maleki et al. (2000) found that roasted peanut proteins bound to IgE from patients with peanut allergy at approximately 90-fold higher levels than the raw proteins. The proposed explanation for this enhancement of IgE binding is the glycation of major allergens to form advanced glycation end-products (AGE) via the Maillard Reaction. More specifically, it was previously found that roasting the peanut at very high temperatures greater than 130° C. resulted in a reduction of IgE binding to Ara h 1 and 3, but an increase in binding to Ara h 2/6 via Western blot experiments.

One other major study to date has investigated the effect of heat and pressure treatments on peanut allergenicity. Cabanillas et al. (2012) demonstrated that peanut-specific IgE binding, as well as the detection of major allergens Ara h 1, 2, and 3, can be reduced by autoclaving roasted peanuts. This was explained by the observation that autoclaving resulted in a decrease of α-helix content and an increase in random coils and/or loops as a function of autoclave pressure and duration as shown by circular dichroism (CD) experiments. Similar decreases in specific IgE binding have been observed when autoclaving other legumes such as lupine allergens and green pea.

NMR spectroscopy is a powerful technique that allows for obtaining a characteristic signature of any given sample, requiring minimal sample preparation. Given the suspected important role of protein glycation by reducing sugars via the Maillard Reaction in the enhancement of the allergic response, ¹H NMR spectroscopy was the analytical technique of choice to develop snapshots of the small molecule profiles of the peanut under different processing conditions (FIG. 4). It focused on the soluble peanut components, and thus analyzed peanut-soaked solutions using solution ¹H NMR. The results showed that, in line with the other experiments reported, high-pressure autoclaving causes a decrease in the amount of intact protein in the peanut. This can be demonstrated by the size and broadening of the peaks in the methyl (0.5-1.5 ppm) and amide (7.0-8.5 ppm) regions of the spectra. The larger peptide molecules leaching out throughout the soaking process tumble more slower in solution, which results in broader peaks in the NMR spectrum, indicating that the autoclaved peanut sample indeed contains a larger number and size of peptide molecules than that of the raw and roasted solutions.

However, other data reported here indicate that certain allergens are being degraded to a greater degree compared to others. Despite using a range of protein extract concentrations three orders of magnitude higher for Ara h 8 compared to Ara h 2, no trace of Ara h 8 was detected by ELISA in the autoclaved extract (FIG. 2B). This is in contrast to a 50% reduction in detection of Ara h 2 in the autoclaved extract when compared to the raw or roasted (FIG. 2A). This suggests that Ara h 2 may be more resistant to digestion via autoclave treatment than Ara h 8.

These differential levels of detection of both Ara h 2 and Ara h 8 may be part of the explanation of the results we observed from the SPT (Table 2). One potential hypothesis is that the patients in the highly allergic group, experiencing severe allergic reactions, have a majority of peanut-specific IgE to Ara h 2, while those in the mildly allergic group, experiencing only oral symptoms to peanut, have most of their peanut-specific IgE to Ara h 8, a plant panallergen cross-reactive with birch pollen protein Bet v 1. Thus, the autoclaved peanut extract which partially contained Ara h1 and Ara h 2, but substantially lacked Ara h 8 (could not be detected), has the potential to be used in an improved diagnostic method for peanut allergy (Table 4).

TABLE 4
Expected outcomes of an improved diagnostic
method using both the whole, standard
peanut extract and autoclaved peanut protein extract.

| | SPT Result | |
|---|---|---|
| | Whole (non-Autoclaved) | Autoclaved |
| Reaction to Peanut | Extract | Extract |
| Risk for anaphylaxis; | ( + ) | ( + ) |
| IgE specific for Ara h 2 | | |
| Allergy dominated | ( + ) | ( − ) |
| by oral | | |
| symptoms; | | |
| IgE specific for Ara h 8 | | |
| Tolerant | ( − ) | ( − ) |

Without wishing to be bound to theory, it is possible that the results observed are the outcome of a dose-related phenomenon. It is known that the Ara h 2 allergen exists in high proportions relative to others, and this is in line with the detection levels of each allergen in FIG. 2 when quantifying the amount of Ara h 2 relative to Ara h 8.

All data reported here support the notion that autoclaving the peanut results in the degradation of its proteins into smaller peptides and free amino acids. Protein extract concentrations of each condition were determined using a Bradford assay, which has a lower detection limit of peptides of size 3 to 5 kilodaltons or greater. When equal weights of dry, defatted peanut flour of each condition was dissolved in the extraction buffer, the Bradford assay produced concentration values for the autoclaved peanut extracts one order of magnitude lower than that of the raw extracts (Table 1). In the Western blot experiments, no clear or distinct bands were detected, but rather a general smear of proteins and peptides (FIG. 1). In the anti-Ara h ELISA experiments, we see that certain protein allergens, such as Ara h 8, degrade to the point of no detection whereas others, such as Ara h 2, still have levels of detectable, intact protein (50% compared to raw, FIG. 2). This is in line with the peanut-specific IgE-binding data as well; with less intact protein allergens, we expect lower IgE binding as a result (FIG. 3). The solution ¹H NMR spectra support this idea as well, demonstrated by the presence of broad peaks in the autoclaved spectrum, corresponding to larger peptide molecules (FIG. 4). Lastly, when assessing IgE binding in vivo via SPT, we observed smaller wheal sizes using the autoclaved extracts when compared to raw, particularly in the patient group that only experience oral symptoms to peanut (Table 2).

The technique of Oral Immunotherapy (OIT) as a treatment for food allergy has been the topic of much research and debate over the past decades, particularly in the context of its safety and efficacy. Some of the safety concerns have been mitigated with similar levels of efficacy when using a processed form of the allergen-containing food as a substrate for OIT. In the case of egg or milk allergies, natural history studies have indicated that patients are able to tolerate increasingly larger amounts of the cooked form of the allergen into their diets and ultimately a significant number evolve to complete tolerance.

In contrast, simply heating the peanut via roasting does not denature its allergens. Current peanut OIT trials using conventional raw or roasted peanuts have shown some success in inducing desensitization and modulating the allergic response. However, high rates of adverse reactions, including anaphylaxis, have been reported and thus peanut OIT cannot be provided without significant levels of risk. One approach currently being tested uses boiled peanut to reduce the allergenicity compared to raw or roasted peanut. Preliminary data suggest that boiled peanut OIT might be a safe and effective substrate to induce desensitization in children with peanut allergy given that boiling appears to result in the loss of key allergenic components (especially Ara h 1, 2 and 6) into the cooking water. The current data reported here demonstrate that high-pressure and temperature autoclaving has the potential to improve the safety and efficacy of peanut OIT by reducing the proportion of intact allergens.

Altogether, the data reported in this example suggest that high-pressure and temperature autoclaving reduces the amount of intact proteins in the peanut, including allergenic proteins. It is currently unknown whether the autoclaving parameters can be optimized to a degree of complete reduction of intact allergens.

### Example II—NMR Characterization of a Processed Peanut Preparation

Chemicals. 3-(Trimethylsilyl-propionic-2,2,3,3-d₄) acid (TSP-d₄) and deuterium oxide (D₂O) were purchased from Sigma-Aldrich.

Sample Physical Processing. Commercially purchased peanuts (Montreal Food Store, Canada) were purchased raw and shelled. Peanuts were roasted with their seed coating in a convection oven at 150° C. for 30 minutes or were autoclaved in a tabletop autoclave at 136° C. at 2.48 Bar (36 psi) for 30 minutes. Analyses were performed in comparison with raw peanut (unprocessed).

Defatting into flour. Raw, roasted and autoclaved peanuts (6 to 12 of each) were ground into a smooth paste using a coffee grinder. The smooth paste was then suspended in hexanes and the peanut flour was collected by filtration under vacuum.

NMR Sample Solid Preparation. Small pieces (6 mg) of whole, intact peanut or defatted peanut flour (4 mg) collected from raw, roasted or autoclaved peanuts were loaded into a KeI-F, disposable insert, which was subsequently placed inside a reusable 4 mm rotor.

NMR Sample Solution Preparation. Six whole peanuts of each condition were placed in 10 mL of double distilled water in a 15 mL falcon tube. The peanuts were allowed to soak in the water at room temperature for 48 hours. Three samples per condition (1 mL each) were evaporated under a SpeedVac at 45° C. for 1.5 hours and the resulting residue reconstituted in 0.6 mL of double distilled water. The three samples were combined together to give a total volume of 1.8 mL, 450 μl of which was collected for analysis.

High-Resolution Magic Angle Spinning ¹H Nuclear Magnetic Resonance Spectroscopy (HR-MAS ¹H NMR). 15 μl of 5 mM TSP-d₄ in 100% D₂O was added to the rotor as an internal standard set to 0.0 ppm prior to the addition of the insert. Analysis was performed in a Bruker 600 MHz NMR equipped with an advanced HR-MAS probe using the water suppression pulse sequence, zgpr (Bruker standard sequence). 64 scans were acquired with an acquisition time of 0.97 s and a spectral width of 8.4 kHz.

Solution ¹H NMR of peanut-soaked solutions. Solution ¹H NMR spectra were run on a Bruker 400 MHz NMR spectrometer for analysis using the Car-Purcell-Meiboom-Gill (CPMG) pulse sequence⁵. 32 scans were acquired with an acquisition time of 3 s and a spectral width of 12 kHz. The ¹H chemical shifts were internally referenced by adding 0.5 mM of TSP-d₄ set to 0.0 ppm.

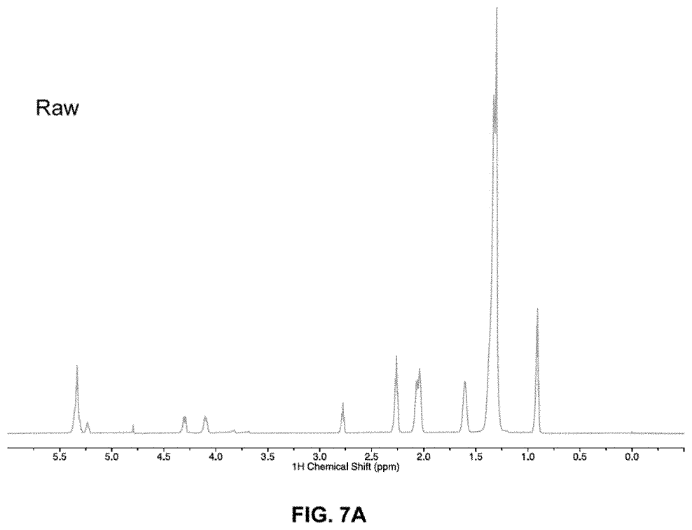

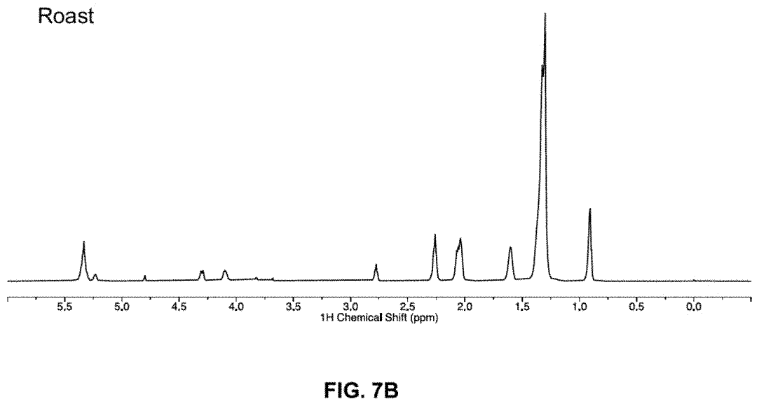

The recent advances in solid state NMR has led to the development of an approach termed High-Resolution Magic-Angle-Spinning (HR-MAS) that allows the analysis of small molecules in the context of semi-solid structures. This has made it an ideal technology to read the small molecule composition of food products in situ. Interestingly, the present example is the first to apply HR-MAS to the analysis of intact peanut. This led to the detection of fatty acid as the largely dominant species of the peanut. Based upon the NMR spectrum, the fatty acids observed are primarily triglycerides with protons at 4.1 and 4.3 ppm, corresponding to the glycerol structure, and at 5.3 ppm corresponding to the unsaturated chains (FIG. 7). The intensity of the peaks observed masked the presence of other small molecules. However, the HR-MAS snapshot did confirm fatty acids in the form of triglycerides as the primary chemical composition of peanuts. As expected, analysis of peanut processed under roasted conditions did not show a significant difference in the lipid profile.

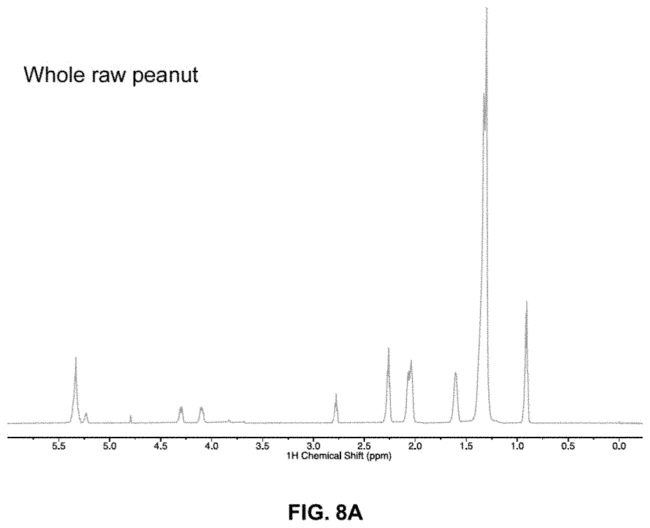

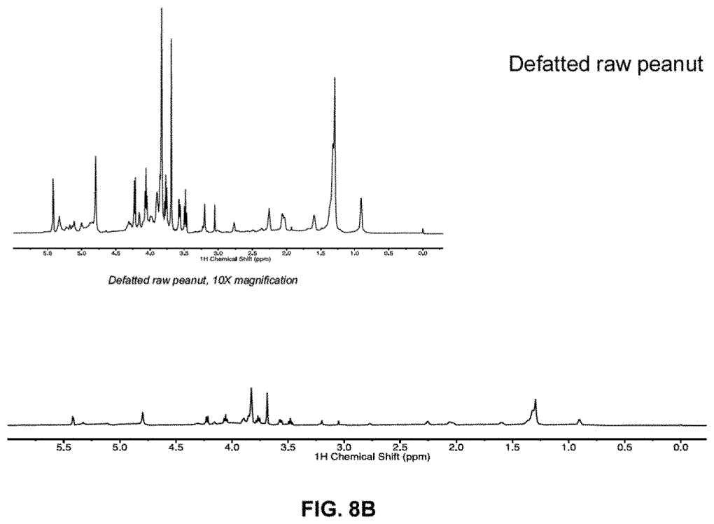

In order to be able to detect other small molecules present in lower proportion in the solid peanut, the samples were defatted using suspensions in hexane. Analysis of the resulting peanut flour showed a marked decrease in lipid-corresponding peaks and appearance of a doublet at 5.4 ppm indicating the anomeric proton of a carbohydrate and a triplet at 3.5 ppm suggesting the presence of sucrose (FIG. 8). This is in agreement with sucrose being one of the dominant small molecules of peanut composition. Barely detectable peaks at 5.2 ppm suggest the presence of glucose.

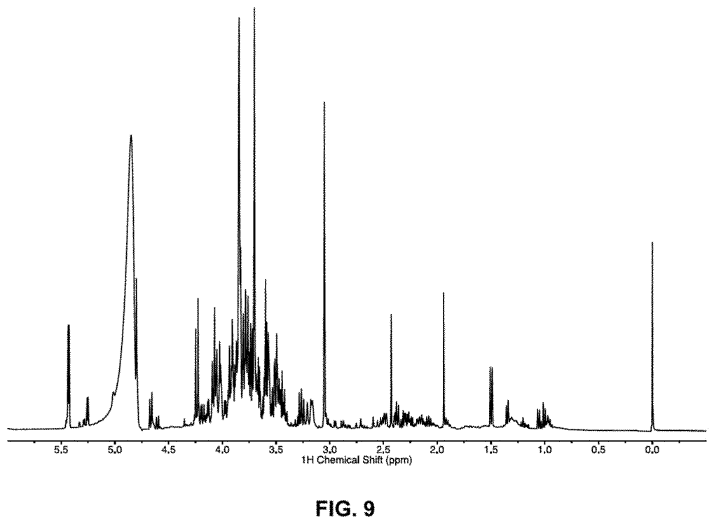

In order to obtain a full picture of soluble molecules of the peanut, it was allowed to soak in water under raw, roasted and autoclaved conditions for 48 hours and the resulting solution analyzed by solution NMR (FIG. 9). NMR analyses were performed to determine whether a differential profile could be observed in the soluble small molecule profiles under the various processing conditions. As expected, the results showed no trace of lipid, but a dominance of the peaks corresponding to sucrose and minor peaks which may correspond to the two anomers of D-glucose at 4.65 ppm and 5.25 ppm.

Analysis of the different areas of the spectrum show the virtually unchanged relative distribution of the sucrose-corresponding peaks. However, in the regions below 2 ppm and from 6.5 to 8.5 ppm, a marked difference was observed in the number of signals in the autoclaved spectrum when compared with the raw and roasted spectra. These regions likely correspond to the methyl hydrogens in the side chains of amino acids or sugars such as rhamnose, and amide hydrogens in peptide backbones and amino acid side chains, respectively. Interestingly, the spectra of roasted conditions resembled more those observed in the raw than the autoclaved spectrum (FIG. 10).

The steady increase in peanut allergy in children has catapulted the topic into intense investigation over the past 5 years. Recent evidence suggests a change in the allergenic properties of the peanut following processing. Glycation, which results from the addition of reducing sugars to the protein side chains in a reaction called the Maillard Reaction, has been proposed as the mechanism of enhancement of allergenic response. Since the reaction occurs between soluble molecules and sugars at high temperatures, it was surmised that the small molecule distribution profile of the peanut could be used to develop signatures for predicting processed-related allergenicity. Therefore, changes in small molecule contents of peanuts under intact structures were analyzed using HRMAS NMR and from solutions of soaked legumes. Interestingly, under the intact structure, dominant structural lipid composition were observed. Indeed, chemical composition analysis using different techniques showed that peanut has a 50% composition of lipid, which is largely dominated by triglycerides. In the present example, this was confirmed by the clear appearance of unsaturated peaks in the 5.3 ppm region, and very intense CH peaks in the 1-2 ppm region. As expected, no significant change in lipid composition profile following roasting conditions was observed in intact peanuts. The triglyceride profile remained intact, which is in agreement with the oily composition of peanut butter, a product manufactured following the high-temperature roasting process of the peanut.

It is noteworthy that removal of the lipids unveiled the presence of the water-soluble molecules in the defatted peanut flour in HRMAS analyses. The spectra were largely dominated by sucrose with a clearly visible doublet of the glucopyranose anomeric proton at 5.4 ppm as well as the doublet and triplet corresponding to the fructose moiety of sucrose at 4.21 ppm and 4.05 ppm, respectively. Interestingly, the major small molecules observed in the spectra with the defatted peanut in the solid resembled those observed in the soaked solution of peanuts, whether they were raw, roasted or autoclaved.

Based upon the results observed by NMR, it appears that two significant classes of molecules are abundantly present in peanuts: triglycerides and sucrose. Indeed, under all conditions studied, a small amount of free glucose was observed through the peak at 4.65 ppm (β-anomer) and 5.25 ppm (α-anomer). The proportion of NMR-detectable sucrose (semi-solid or liquid) was largely superior to that of glucose. These results suggest that modifications of the proteins (e.g., allergens) in the peanut may occur between a small fraction of free molecules available in the peanut legume. However, no direct Maillard Reaction-type reaction with amine side chain has been reported.

Moreover, the fact that sucrose is a non-reducing sugar means that it can only contribute to the Maillard Reaction following its conversion into glucose and fructose. It is thus also a possibility that sucrose molecules present in the peanut in high proportions may be hydrolyzed into glucose and fructose at high temperatures, of which glucose molecules immediately react with amine side chains, leaving a similar baseline level of glucose detection by NMR despite the manifestation of the Maillard reaction.

More interestingly, based upon the NMR results, the changes in small molecule profiles under different conditions appear to affect a small fraction of molecules, as can be seen in the regions below 2 ppm and from 6.5 to 8.5 ppm, which contain a significant level of broad signals in the autoclaved spectra versus the raw or roasted spectra. This broadening effect may be due to the breaking down of proteins into short peptides and amino acids throughout the autoclaving process, thus being more readily released into solution throughout soaking and revealing them in the NMR analysis. The results demonstrate that changes in allergenicity brought about by small molecules (e.g., the Maillard Reaction) may be performed with a small fraction of the molecular content of the entire peanut. Further studies are ongoing to correlate these changes with allergenic response.

While the invention has been described in connection with specific embodiments thereof, it will be understood that the scope of the claims should not be limited by the preferred embodiments set forth in the examples, but should be given the broadest interpretation consistent with the description as a whole.

### REFERENCES

Cabanillas, B.; Maleki, S. J.; Rodriguez, J.; Burbano, C.; Muzquiz, M.; Jimenez, M. A.; Pedrosa, M. M.; Cuadrado, C.; Crespo, J. F., Heat and pressure treatments effects on peanut allergenicity. Food Chem 2012, 132 (1), 360-6.
Maleki, S. J.; Chung, S.-Y.; Champagne, E. T.; Raufman, J.-P., The effects of roasting on the allergenic properties of peanut proteins. Journal of Allergy and Clinical Immunology 2000, 106 (4), 763-768.
Walczyk, N. E.; Smith, P. M. C.; Tovey, E. R.; Roberts, T. H., Peanut protein extraction conditions strongly influence yield of allergens Ara h 1 and 2 and sensitivity of immunoassays. Food Chem 2017, 221, 335-344.

What is claimed is:

1. A process for making a peanut hypoallergenic formulation:
(a) providing an initial composition comprising a Ara h 2 allergen and a Ara h 8 allergen; and
(b) submitting the initial composition to a simultaneous heat treatment and pressure treatment to obtain the peanut hypoallergenic formulation, wherein the peanut hypoallergenic formulation substantially lacks the Ara h 8 allergen.

2. The process of claim 1, wherein the initial composition comprises or is derived from a whole peanut.

3. The process of claim 1, wherein the heat treatment comprises heating the initial composition to a temperature between 120° C. and 140° C.

4. The process of claim 1, wherein the pressure treatment comprises applying a pressure between 1.15 and 2.60 atm and/or a vapor pressure to the initial composition to the initial composition.

5. The process of claim 4, wherein step (b) comprises autoclaving the initial composition.

6. The process of claim 1 further comprising:
cooking, pureeing, extruding and/or making a flour of the initial composition, the peanut hypoallergenic formulation, or both;
formulating the peanut hypoallergenic formulation in a skin prick test reagent, an oral immunotherapy reagent or a food product; and/or
performing a ¹H nuclear magnetic resonance (¹H NMR) analysis of the initial composition, the peanut hypoallergenic formulation or both.

7. A peanut hypoallergenic formulation comprising a Ara h 2 allergen and substantially lacking a Ara h 8 allergen.

8. The peanut hypoallergenic formulation of claim 7 having a ¹H nuclear magnetic resonance (¹H NMR) spectrum profile at least substantially similar or identical to the ¹H NMR spectrum profile shown in FIG. 10C in the range between 0.5 and 2 ppm and between 6.5 and 8.5 ppm.

9. The peanut hypoallergenic formulation of claim 7 being obtained by the process of claim 1.

10. A method for stratifying the risk of a subject to develop anaphylaxis upon the ingestion of a peanut allergen, the method comprising:
pricking the skin of the subject at a first location to make a first skin abrasion;
contacting a first reagent with the first skin abrasion, wherein the first reagent comprises a Ara h 2 allergen and substantially lacking or devoid of a Ara h 8 allergen; and
determining if the first reagent caused a wheal and flare response towards the first reagent;
wherein the presence of the wheal and flare response towards the first reagent is indicative that the subject has an increased risk of developing anaphylaxis upon the ingestion of the peanut allergen when compared to a first control subject that did not exhibit a wheal and flare response towards the first reagent.

11. The method of claim 10, further comprising:
pricking the skin of the subject at a second location to make a second skin abrasion;
contacting a second reagent with the second skin abrasion, wherein the second reagent comprises a Ara h 8 allergen; and
determining if the second reagent caused a wheal and flare response towards the second reagent;
wherein the presence of the wheal and flare response towards the second reagent, but not towards the first reagent, is indicative that the subject has a decreased risk of developing anaphylaxis upon the ingestion of the peanut allergen when compared to a second control subject that exhibited a wheal and flare towards to the first reagent.

12. The method of claim 11, wherein the subject was previously determined to exhibit a wheal and flare response to the second reagent.

13. The method of claim 10 for screening for subjects suitable to start and/or continue an oral immunotherapy.

14. The method of claim 10 further comprising administering an oral immunotherapy to the subject whose risk to develop anaphylaxis has been stratified.

15. The method of claim 10, further comprising orally administering an oral immunotherapy reagent or a food product of claim to the subject, wherein the oral immunotherapy agent and the food product comprises a peanut hypoallergenic formulation comprising a Ara h 2 allergen and substantially lacking a Ara h 8 allergen.
